(19) United States
(12) Patent Application Publication
(10) Pub. No.: US 2010/0228155 A1
(43) Pub. Date: Sep. 9, 2010

(54) MUSCULAR STRENGTH TEST SYSTEM

(76) Inventor: Jae Lak Yang, Busan (KR)

Correspondence Address: CHRISTOPHER PAUL MITCHELL, 888 16TH ST., NW, SUITE 800, WASHINGTON, DC, 20006, US

(21) Appl. No.: 12/680,573

(22) PCT Filed: Sep. 22, 2008
(86) PCT No.: PCT/KR08/05622

§ 371 Date: Mar. 28, 2010

(30) Foreign Application Priority Data
Sep. 28, 2007 (KR) 10-2007-0098151

(51) Int. Cl. A61B 5/103

(52) U.S. Cl. 600/587

(57) ABSTRACT

Disclosed therein is a muscular strength test system, which can test muscular strength of a specific part of the body in a state where a user binds the body to the frame in safety. The muscular strength test system includes: a controlling part (2); a shaft (4) rotatably connected to a motor (3) via electromotive means (3a); an angle-adjustable frame (5) mounted on an end portion of the shaft (4), the angle-adjustable frame (5) comprising a main frame (5a) adjusting its angle back and forth by the motor (3), and a sub frame (5b) mounted on a motor (6) of the main frame (5a) via a shaft (7); a support frame (5c) vertically mounted on the sub frame (5b); leg binding means (8), hip binding means (9), and head binding means (10) mounted on the support frame (5c) to selectively bind the user's legs, hips and head to the support frame (5c); and sensing means (11) mounted at right and left sides of the sub frame (5b) for sensing muscular strength.

### TECHNICAL FIELD

The present invention relates to a muscular strength test system, and more particularly, to a muscular strength test system, which can allow a user to test muscular strength of a specific part of the body and correct a poor posture in taking an exercise by giving an inclination to the user's body in such a way that gravity acts to the user's body in a state where the user binds his or her body to the frame in safety, thereby providing data of muscular strength, a guideline for correcting the poor posture, and a correct exercise prescription.

### BACKGROUND ART

While there are many exercise machines to exercise a specific part of a user's body using gravity, there is no means for testing muscular strength of the specific part of the body.

That is, the conventional exercise machines are to strengthen erector spine, which supports the waist, after the user adjusts an angle of an angle-adjustable frame in such a way as to have a predetermined slop in a state where the user binds the legs and the hips of the lower part of the body to the angle-adjustable frame.

However, the conventional exercise machines have several problems in that they have no means for reinforcing muscular strength of the upper part of the body, and in that it is difficult to provide a correct exercise prescription since it cannot test muscular strength of an objective numerical value when the user takes an exercise to strengthen muscular strength around the waist in a state where the frame is inclined.

### DISCLOSURE OF INVENTION

### Technical Problem

Accordingly, the present invention has been made in an effort to solve the above-mentioned problems occurring in the prior arts, and it is an object of the present invention to provide a muscular strength test system for testing muscular strength using gravity, which includes sensing means for sensing a user's poor posture and transferring the sensed result to a controlling part when the muscular strength lowers after the user adjusts an angle of an angle-adjustable frame to a predetermined slop in a state where the user binds the upper half and the lower half of the body to the angle-adjustable frame in safety, thereby testing the user's muscular strength and correcting the user's poor posture.

### Technical Solution

To achieve the above objects, the present invention provides a muscular strength test system, which can test muscular strength of a specific part of the body in a state where a user selectively binds the upper and lower parts (namely, the legs, the hips, the head, and so on) of the body to an angle-adjustable frame using binding means and selectively grasps support frames of the angle-adjustable frame with the hands, thereby providing a correct exercise prescription by testing muscular strength in a poor posture after a predetermined time period in the state where the upper or lower part of the body is bound to the angle-adjusted frame.

### ADVANTAGEOUS EFFECTS

The muscular strength test system according to the present invention can provide data of muscular strength, an exercise guideline and a scientific muscle-strengthening exercise plan by allowing muscular strength test of the specific part of the body and correction of the poor posture in taking an exercise since the angle-adjustable frame can be inclined to a predetermined slop back and forth or right and left in such a way that gravity acts to muscles of the specific part of the body in a state where the user's body is bound to the angle-adjustable frame stably, thereby greatly contributing to national health.

### BRIEF DESCRIPTION OF THE DRAWINGS

FIG. 1 is a front view of a muscular strength test system according to a first preferred embodiment of the present invention.

FIG. 2 is a side elevation view showing an operational state of the muscular strength test system according to the first preferred embodiment of the present invention.

FIG. 3 is a side elevation view showing an operational state of the muscular strength test system according to the first preferred embodiment of the present invention.

FIG. 4 illustrates a front view showing a state where sensing means are mounted on the muscular strength test system and an enlarged plan view of essential parts of the muscular strength test system.

FIG. 5 is a side elevation view showing an operational state where the sensing means are mounted on the muscular strength testing system.

FIG. 6 illustrates a front view of a muscular strength test system according to a second preferred embodiment of the present invention and a perspective view of essential parts of the muscular strength test system.

FIG. 7 is a side elevation view showing an operational state of the muscular strength test system according to the second preferred embodiment of the present invention.

FIG. 8 is a plan view showing a state where sensing means and a guide frame are mounted to the muscular strength test system according to the second preferred embodiment of the present invention.

FIG. 9 is a front view showing an operational state of the muscular strength test system according to the second preferred embodiment of the present invention.

FIG. 10 illustrates a front view showing another example of the sensing means mounted on the muscular strength test system according to the second preferred embodiment of the present invention and a plan view of essential parts thereof.

FIG. 11 illustrates a front view showing a further example of the sensing means mounted on the muscular strength test system according to the second preferred embodiment of the present invention and a plan view of essential parts thereof.

### MODE FOR THE INVENTION

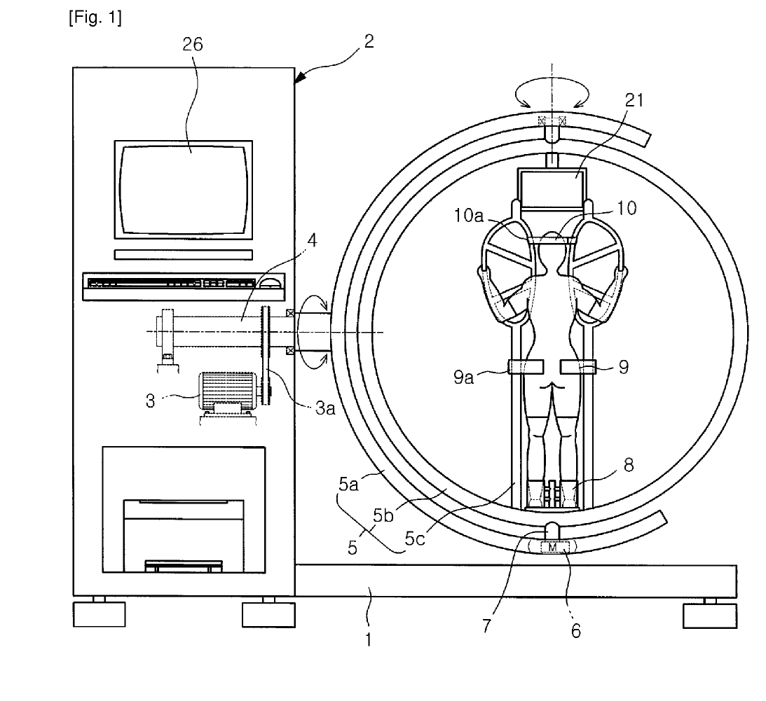

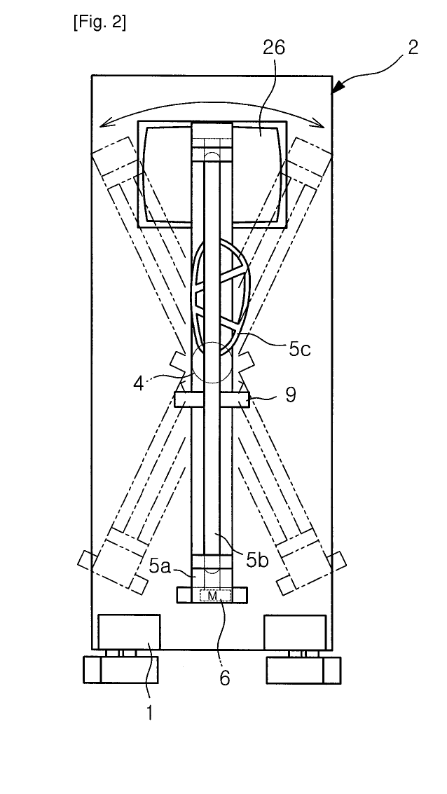

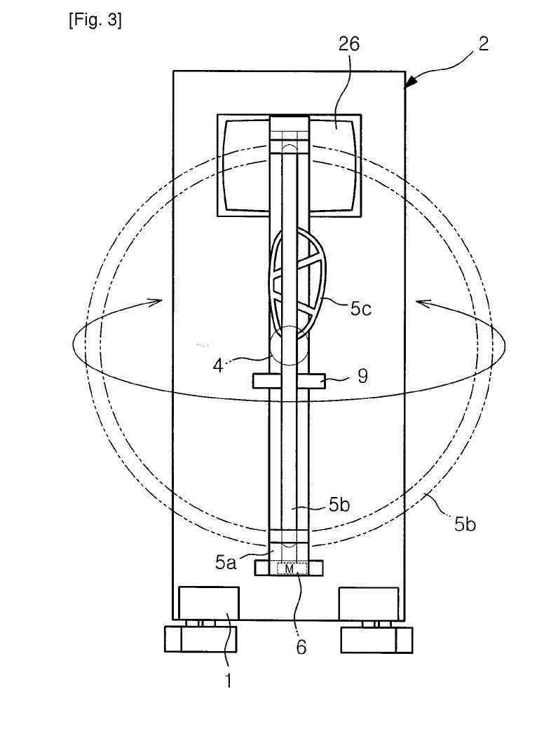

Referring to FIGS. 1 to 5, a muscular strength test system according to the present invention will be described. That is, the muscular strength test system includes a base frame 1, a controlling part 2 mounted on a side of the base frame 1 or mounted separately, a motor 3 embedded in the controlling part 2, a shaft 4 connected with the motor 3 via electromotive means 3a and rotatably shaft-mounted on the motor 3 in a horizontal direction, and an angle-adjustable frame 5 mounted on an end portion of the shaft 4 in such a way as to adjust its angle back and forth as shown in FIG. 1 according to a forward and backward driving of the motor 3.

The frame 5 includes a main frame 5a formed in an approximately arc shape and adjusting its angle back and forth by the motor 3, and a sub frame 5b connected to a motor 6 mounted on the main frame 5a via a shaft 7 to thereby control right and left rotation on the main frame 5a.

Furthermore, a support frame 5c is vertically mounted on the sub frame 5b, and then, leg binding means 8, hip binding means 9 and head binding means 10 are respectively mounted on the sub frame 5b, so that a user can stably bind a specific part of the body to the frame 5 in a state where the user stands erect.

Since the leg binding means 8 and the hip binding means 9 are selected from commonly used exercise machines, a detailed description of the leg binding means 8 and the hip binding means will be omitted.

Moreover, it is preferable that the hip binding means 9 and the head binding means 10 respectively have ring-type frame binding portions 9a and 10a ascending and descending on the support frame 5c, so that the hip binding means 9 and the head binding means 10 can be adjusted in height according to users' body sizes.

An upper portion of the support frame 5c is constructed in such a way that the user can selectively grasp the support frame 5c with the hands and the upper portion of the support frame 5c can support the user's back or breast in case of need, whereby the user can test muscular strength with a sense of stability.

Sensing means 11 for sensing that the user's body is deviated from a correct position due to a lowering of the muscular strength are mounted at right and left sides of the sub frame 5b.

It is preferable that the sensing means 11 are at least one photo sensor 12 and 13. Since sensed positions are different according to the users' body sizes, each of the sensing means includes a sensor frame 14 having a guider 14a formed in such a way that the sensing means 11 can be vertically controlled on the sub frame 5b, whereby the sensing means 11 can be vertically adjusted in its height along a guide groove 14b on the sub frame 5b. Of course, after the height of the sensing means 11 is adjusted, in order to fix the height of the sensing means 11, the guider 14a and the guide groove 14b are constructed in such a way as to be tightly slid by an external force, so that the adjusted position can be kept when the external force is removed. Alternatively, the user can fix the adjusted position using a separate adjustable bolt (not shown). That is, various kinds of position adjusting and fixing means can be applied.

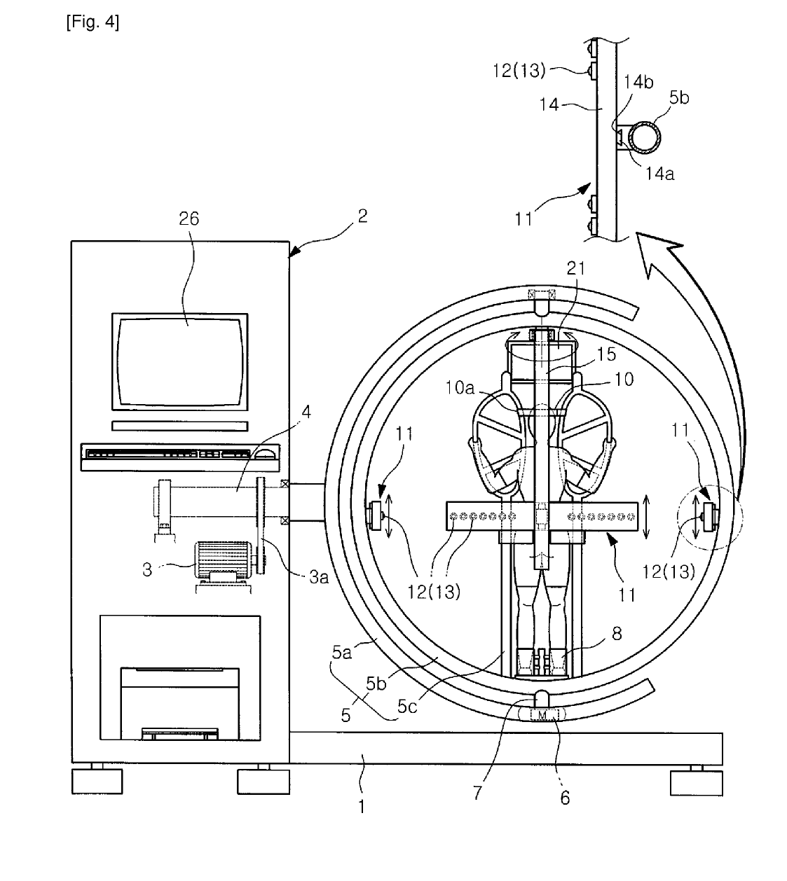

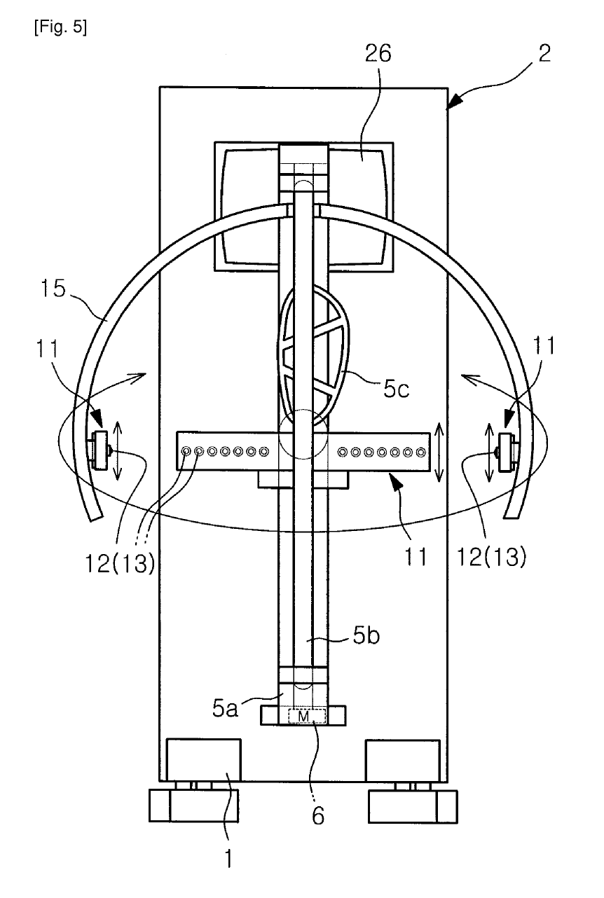

In order to sense that the user's body deviates from the correct position in right and left directions when he or she is seen from the front, as shown in FIGS. 4 and 5, a supporter 15 is mounted on the sub frame 5b in an arc form in a back and forth direction, and then, the sensing means 11 as described above may be mounted thereon.

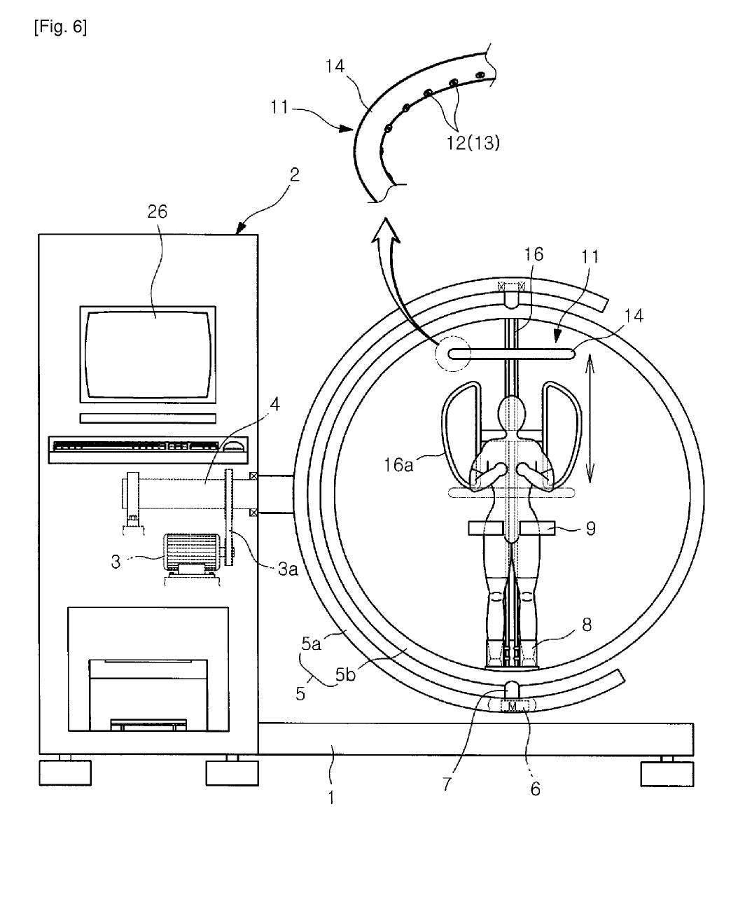

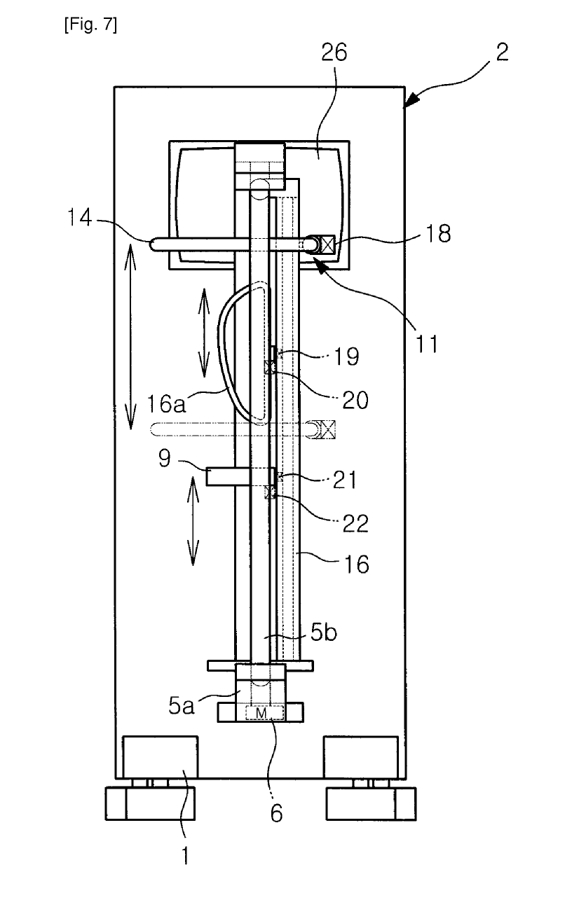

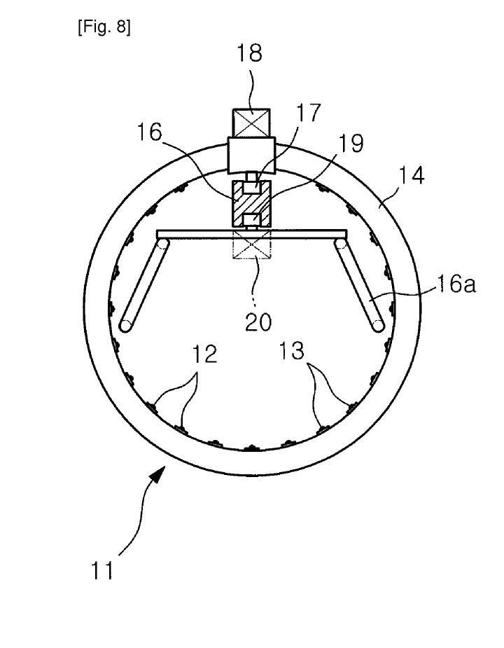

FIGS. 6 to 8 illustrate a muscular strength test system according to a second preferred embodiment of the present invention, which includes sensing means 11 selectively usable when muscular strength of the upper part of the body is tested.

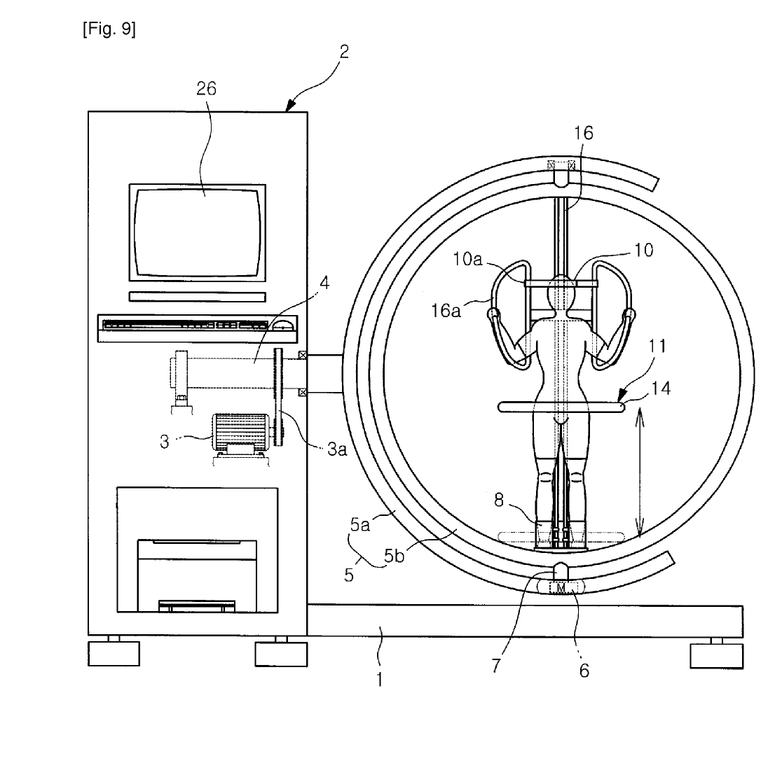

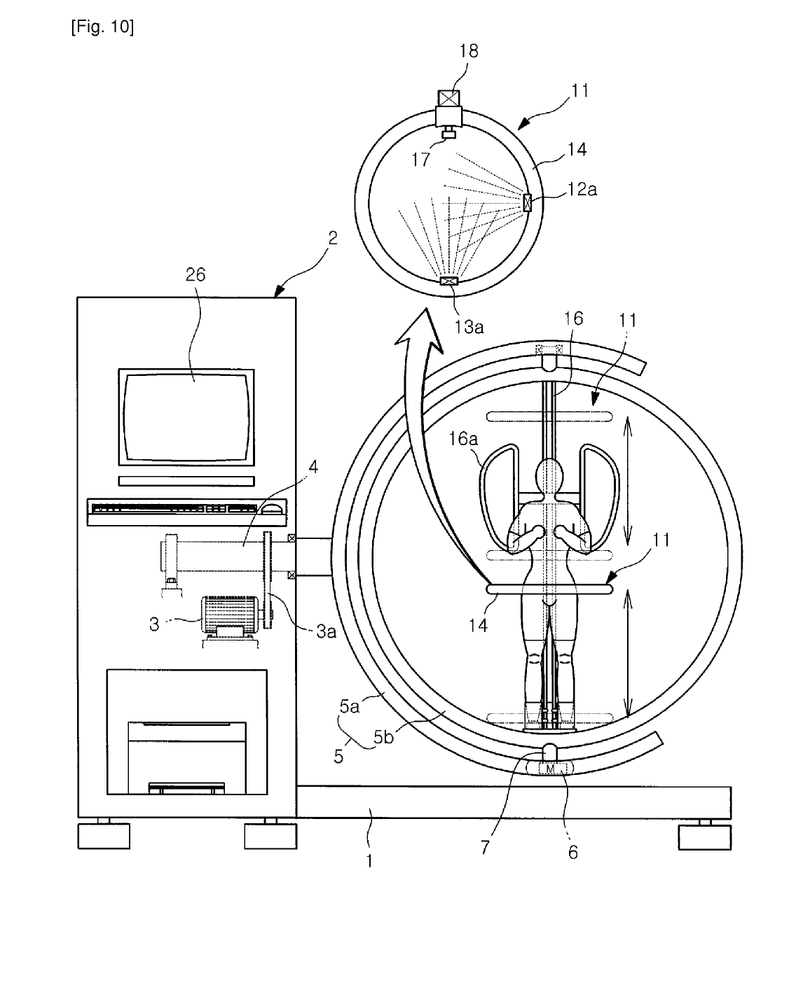

FIGS. 9 and 10 illustrate a used state of the sensing means 11 when muscular strength of the lower part of the body is tested using the sensing means 11, and it will be described in more detail as follows.

That is, the sensing means 11 includes a guide frame 16 fixed on the rear face of the sub frame 5b and having a cross section of an approximately H-form, a linear motor guider 17 mounted on the rear face of the guide frame 16, and a ring-shaped sensor frame 14 ascending and descending along the linear motor guider 17 by a driving of a motor 18.

In addition, the sensor frame 14 has a plurality of photo sensors 12 and 13 mounted at predetermined intervals to thereby measure the muscular strength of the upper and lower parts of the body.

As shown in FIG. 10, the photo sensors 12 and 13 may include at least one distance meter 12a and 13a using laser or infrared rays.

The guide frame 16 includes support frames 16a mounted on an upper portion of the front face thereof for allowing the user to grasp them with the hands to support the upper part of the body, and the support frames 16a ascending and descending along a linear motor guider 19 by a driving of a motor 20.

Furthermore, the hip binding means 9 for binding the user's hips to the frame is mounted on a lower portion of the front face of the guide frame 16 in such a way as to ascend and descend along a linear motor guider 21 by a driving of a motor 22 to thereby be compatible according to the users' body sizes.

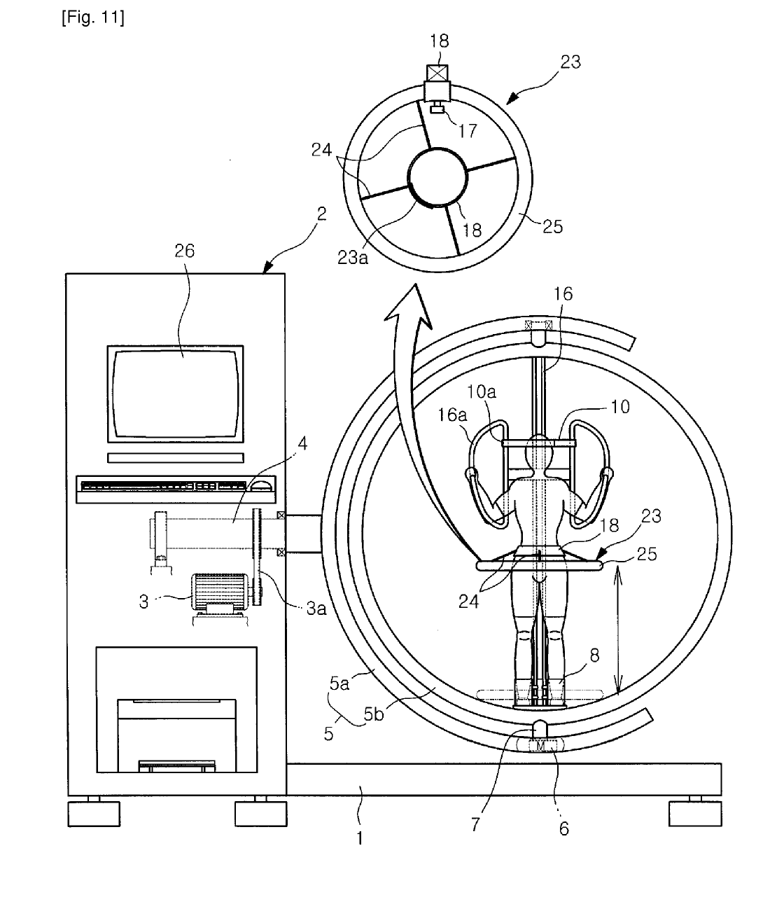

FIG. 11 illustrates another preferred embodiment of the present invention. In FIG. 11, the sensing means 11 are mounted on the user's hips using a belt 23 having detachably attaching means 23a such as a magic tape. Accordingly, the user can test muscular strength of the body using the belt 23 and a ring-type sensing bar 25 radially connected with the belt 23 via ropes 24.

In the drawing, the unexplained reference numeral 26 indicates a monitor.

The muscular strength test system according to the present invention can prepare an exercise guideline proper to the user's muscular strength by testing muscular strength of a specific part of the body and sensing the poor posture in taking an exercise to thereby allow scientific exercise prescription and medical cure prescription. Hereinafter, a method of using the muscular strength test system according to the present invention will be described in more detail.

First, referring to FIGS. 1 to 5, the user who wants to test his or her muscular strength binds his or her body to the muscular strength test system in safety selectively using the leg binding means 8 mounted on the support frame 5c, the support frame 16a for selectively supporting the arms, the hip binding means 9, and the head binding means 10.

After that, when the controlling part 2 selectively operates the motors 3 and 6 to test the muscular strength, the shafts 4 and 7 are selectively operated in such a way that the main frame 5a is adjusted back and forth to a predetermined angle or the sub frame 5b is adjusted right and left to a predetermined angle, and accordingly, the user's body is inclined to one of back and forth directions or right and left directions. When the muscular strength of each muscle of the body is exhausted at a specific inclination angle, a posture of a specific part of the body gets poor in the initially inclined posture, and the sensing means 11 sense the poor posture and transfers the sensed result to the controlling part 2. The controlling part 2 measures the muscular strength of muscles of the corresponding body part based on the transferred data and stores it into a database. Based on the measured data of the muscular strength, the user can obtain the exercise prescription or the medical cure prescription when there is any error in muscles.

Moreover, as shown in FIGS. 6 to 8, since the sensor frame 14 of the sensing means 11 ascends and descends along the linear motor guider 17 mounted on the guide frame 16 of the sub frame 5b by the driving of the motor 18, the position of the sensing means 11 can be adjusted vertically according to the users' body conditions, whereby the user can test his or her muscular strength conveniently using the various sensing means 11, such as the photo sensors 12 and 13 or the distance meters 12a and 13a.

The support frames 16a mounted on the upper portion of the front face of the guide frame 16 can be also vertically adjusted in height along the linear motor guider 19 by the driving of the motor 20 according to the users' body conditions, whereby the user can test his or her muscular strength in safety in a state where the user grasps the support frames 16a with the hands.

Additionally, the hip binding means 9 mounted on the lower portion of the front face of the guide frame 16 can be also vertically adjusted in height along the linear motor guider 21 by the driving of the motor 22, whereby the user can test his or her muscular strength compatibly according to the users' body sizes.

Referring to FIG. 11, the sensing means 11 are mounted on the user's hips by the belt 23 having the detachably attaching means 23a, and then, the user can test his or her muscular strength using the ring-type sensing bar 25 radially connected to the belt 23 via the rope 24.

### INDUSTRIAL APPLICABILITY

The muscular strength test system according to the present invention can be widely distributed and utilized in various industrial fields related with manufacture of medical instruments, exercise machines, exercise measuring machines, and so on.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

1. A muscular strength test system comprising:
a controlling part 2;
a shaft 4 rotatably connected to a motor 3 via electromotive means 3a in such a way as to be mounted horizontally;
an angle-adjustable frame 5 mounted on an end portion of the shaft 4, the angle-adjustable frame 5 comprising a main frame 5a adjusting its angle back and forth by the motor 3 and a sub frame 5b mounted on a motor 6 of the main frame 5a via a shaft 7 in such a way as to be controlled in its right and left rotation on the main frame 5a;
a support frame 5c vertically mounted on the sub frame 5b;
leg binding means 8, hip binding means 9, and head binding means 10 mounted on the support frame 5c to selectively bind the user's legs, hips and head to the support frame 5c; and
sensing means 11 mounted at right and left of the sub frame 5b for sensing muscular strength of the user's body.

2. The muscular strength test system according to claim 1, wherein the sensing means 11 comprises:
at least one photo sensor 12; 13; and
a guider 14a formed on a sensor frame 14 adapted to vertically adjust height of the sensing means 11 along a guide groove 14b formed on the sub frame 5b.

3. The muscular strength test system according to claim 1, wherein the sensing means 11 is additionally mounted on a supporter 15 formed on the sub frame 5b in a back and forth direction.

4. The muscular strength test system according to claim 1, wherein the sensing means 11 is at least one distance meter 12a; 13a mounted on the sensor frame 14.

5. The muscular strength test system according to claim 1, wherein the sensing means 11 is at least one distance meter 12a; 13a mounted on the sensor frame 14.

6. The muscular strength test system according to claim 1, wherein the hip binding means 9 and the head binding means 10 respectively have ring-type frame binding portions 9a; 10a ascending and descending on the support frame 5c, so that the hip binding means 9 and the head binding means 10 can be adjusted in height according to users' body sizes.

7. A muscular strength test system comprising:
a controlling part 2;
a shaft 4 rotatably connected to a motor 3 via electromotive means 3a in such a way as to be mounted horizontally;
an angle-adjustable frame 5 mounted on an end portion of the shaft 4, the angle-adjustable frame 5 comprising a main frame 5a adjusting its angle back and forth by the motor 3, a sub frame 5b mounted on a motor 6 of the main frame 5a via a shaft 7 in such a way as to be controlled in its right and left rotation on the main frame 5a, and a guide frame 16 mounted on the rear face of the sub frame 5b;
sensing means 11 ascending and descending along a linear motor guider 17 mounted on the rear face of the guide frame 16 by a driving of a motor 18;
a support frame 16a ascending and descending along a linear motor guider 19 mounted on an upper portion of the front face of the guide frame 16 by a driving of a motor 20; and
a hip binding means 19 ascending and descending along a linear motor guider 21 mounted on a lower portion of the front face of the guide frame 16 by a driving of a motor 22.

8. The muscular strength test system according to claim 7, wherein the sensing means 11 comprises a sensor frame 14 and a plurality of photo sensors 12; 13 mounted on the sensor frame 14 at a predetermined interval, whereby the user can selectively test his or her muscular strength of the upper part and the lower part of the body.

9. The muscular strength test system according to claim 7, wherein the sensing means 11 comprises a sensor frame 14 and at least one distance meter 12a; 13a mounted on the sensor frame 14, whereby the user can selectively test his or her muscular strength of the upper part and the lower part of the body.

10. The muscular strength test system according to claim 7, wherein the sensing means 11 comprises:
a belt 23 having a detachably attaching means 23a detachably attached on the user's hips; and
a ring-type sensing bar 25 radially connected with the belt 23 via a rope 24.
